(19) United States
(12) Patent Application Publication
(10) Pub. No.: US 2020/0164010 A1
(43) Pub. Date: May 28, 2020

(54) METHOD FOR TREATING DEMENTIA

(71) Applicant: ARJIL BIOTECH HOLDING COMPANY LIMITED, Hsinchu (TW)

(72) Inventors: Yeh B WU, Hsinchu City (TW); Jir-Mehng LO, Hsinchu City (TW); Hui Ju LIANG, Taipei City (TW); Pei-Hsin LIN, Hsinchu City (TW); Huey-Jen TSAY, Taipei City (TW)

(73) Assignee: ARJIL BIOTECH HOLDING COMPANY LIMITED, Hsinchu (TW)

(21) Appl. No.: 16/699,377

(22) Filed: Nov. 29, 2019

Related U.S. Application Data
(60) Provisional application No. 62/772,211, filed on Nov. 28, 2018.

(51) Int. Cl. A61K 36/07; A61P 25/28

(52) U.S. Cl. CPC A61K 36/07; A61P 25/28

(57) ABSTRACT

The present invention provides a method for treating dementia, in particular Alzheimer's disease, with an extract of Antrodia camphorata fruit body, or active ingredients thereof. Also provided is the use of the extract of Antrodia camphorate or active ingredients thereof for manufacturing a medicament for treating dementia, in particular Alzheimer's disease.

### CROSS REFERENCE TO RELATED APPLICATIONS

This application claims the benefit of and priority to U.S. Provisional Application Ser. No. 62/772,211, filed Nov. 28, 2018, which is incorporated herein by reference in its entirety.

### FIELD OF THE INVENTION

The present invention pertains to a method for treating dementia.

### BACKGROUND OF THE INVENTION

Alzheimer's disease (AD) is characterized by progressive cognitive decline, neurofibrillary tangles, amyloid plaques, neuro-inflammation, and decline of adult neurogenesis (1-3). Amyloid β (Aβ), a peptide formed from procession of amyloid precursor protein (APP), is thought to be one of the primary initiating factors in AD pathology. The amyloid hypothesis recommends that AD is caused by an imbalance between production and clearance of Aβ (1), resulting in increased amounts of Aβ in monomeric, oligomeric and insoluble fibrillary forms in the Central Nervous System (CNS) and which subsequently induces Aβ plaque formation, neuroinflammation and oxidative stress (4).

Increasing evidence has indicated that adult hippocampal neurogenesis play the major role in cognitive function (5). The APP metabolites, including oligomeric Aβ, soluble APPα (sAPP)α and APP intracellular domain (AICD), have been found to regulate the properties of human neural stem cells which influence adult hippocampal neurogenesis (6,7). On the other hand, the proinflammatory cytokines such as IL-1β, TNF-γ, and IL-6 produced by activated glia may also regulate the process of adult hippocampal neurogenesis (8-10).

The APPswe/PS1dE9 mouse model (APP/PS1), co-expressed Swedish, mutated human APP695 and human mutated presenilin 1 (PS1) in which exon 9 is deleted (11), exhibit AD-like pathological and behavioral changes, including accumulation of amyloid plaques in brain, degeneration of cholinergic system, and impaired exploratory behavior and spatial memory (12). Increased Aβ production and plaque formation in APP/PS1 mice has been shown to occur as early as 3- to 5-month-old (13), and impaired spatial learning and memory was observed at 6-month-old (14-15). Furthermore, the neurogenesis is also found to be impaired in the APP/PS1 mouse at 3- to 6-month-old (16).

There remains a need for developing medications for the treatment of AD with no side effect and low toxicity.

### BRIEF SUMMARY OF THE INVENTION

It is an object of the present invention to provide a method for treating dementia comprising administering a subject in need thereof an extract of Antrodia camphorata fruit body as an active ingredient.

In another object, the present invention provides a use of an extract of Antrodia camphorata fruit body for manufacturing a medicament for treating dementia.

Particularly, the extract of Antrodia camphorata fruit body is an extract of a dish cultured fruit body of Antrodia camphorate, hereafter called as ARH003, or an extract of a cutting wood-cultured fruit body of Antrodia camphorate, hereafter called as ARH004.

In one example of the invention, the extract of Antrodia camphorata fruit body is prepared through extraction of Antrodia camphorate fruit body with water or an organic solvent.

In one particular example of the invention, the extract of Antrodia camphorata fruit body comprises one or more compounds as active ingredients, which is selected from the group consisting of the following:

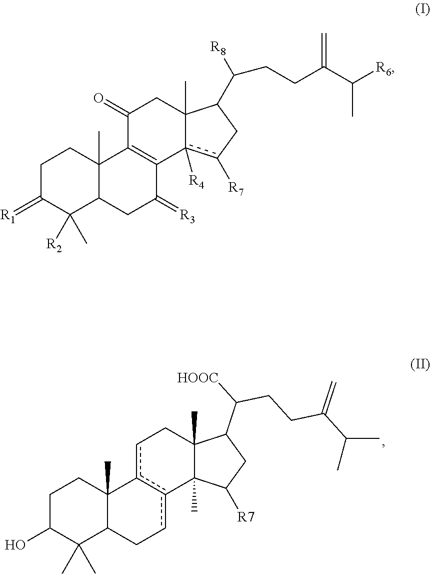

wherein R₁ is O, α-OH or β-H; R₂ is H or OH; R₃ is O, α-H, β-OAc or H₂; R₄ is H or OH; R₅ is H, or OH; R₆ is COOH or COO(CH₂)n-CH₃; n is an integer from 0-3; R₇ is H, OH or OAc; R₈ is CH₃ or COOH; the dotted line represents a single bond or a double bond.

In some particular examples of the present invention, the compound is selected from the group consisting of the following:

dehydroeburicoic acid having the structure of:

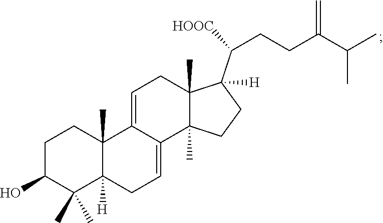

dehydrosulphurenic acid (also called as dehydrosulfurenic acid) having the structure of:

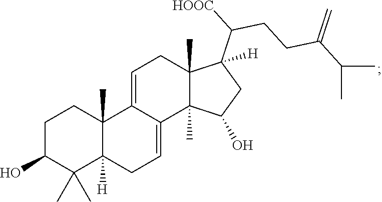 [SMILES: C=C(CCC(C(=O)O)[C@H]1C[C@H](O)[C@@]2(C)C3=CC[C@H]4C(C)(C)[C@@H](O)CC[C@]4(C)C3=CC[C@]12C)C(C)C]

15α-acetyldehydrosulphurenic acid having the structure of:

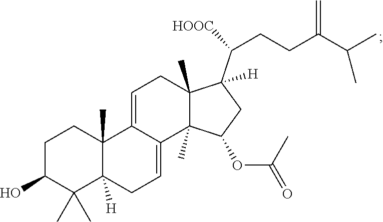 [SMILES: C=C(CC[C@@H](C(=O)O)[C@H]1C[C@H](OC(C)=O)[C@@]2(C)C3=CC[C@H]4C(C)(C)[C@@H](O)CC[C@]4(C)C3=CC[C@]12C)C(C)C]

and antcin K having the structure of:

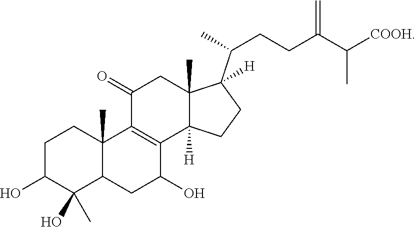 [SMILES: C=C(CC[C@@H](C)[C@H]1CC[C@H]2C3=C(C(=O)C[C@]12C)[C@@]1(C)CCC(O)[C@](C)(O)C1CC3O)C(C)C(=O)O]

On the other hand, the present invention provides a method for treating dementia comprising administering a subject in need thereof one or more of the active compounds as mentioned above.

The present invention also provides the use of the above-mentioned compound for manufacturing a medicament for treating dementia.

In one embodiment of the invention, the dementia is AD.

In one further aspect, the invention provides a method for suppressing the formation of Aβ plaque and glial cells activation comprising administering to a subject in need thereof the extract of Antrodia camphorata fruit body as mentioned above.

Also, the invention provides a use of the extract of Antrodia camphorata fruit body for manufacturing a medicament for suppressing the formation of Aβ plaque and glial cells activation.

In one further aspect, the invention provides a method for suppressing the formation of Aβ plaque and glial cells activation comprising administering to a subject in need thereof one or more of the above-mentioned compounds.

Also, the invention provides a use of the above-mentioned compound for manufacturing a medicament for suppressing the formation of Aβ plaque and glial cells activation.

In one yet aspect, the present invention provides a method for improving memory, comprising administering to a subject in need thereof the extract of Antrodia camphorata fruit body.

Also, the invention provide a use of the above-mentioned compound for manufacturing a medicament for improving memory.

In the invention, the extract or compound according to the present invention provides an efficacy for treating dementia, ameliorating memory impairment and/or improving memory.

It is to be understood that both the foregoing general description and the following detailed description are exemplary and explanatory only and are not restrictive of the invention.

### BRIEF DESCRIPTION OF THE SEVERAL VIEWS OF THE DRAWINGS

The patent or application file contains at least one color drawing. Copies of this patent or patent application publication with color drawing will be provided by the USPTO upon request and payment of the necessary fee.

The foregoing summary, as well as the following detailed description of the invention, will be better understood when read in conjunction with the appended drawings. For the purpose of illustrating the invention, there are shown in the drawings embodiments which are presently preferred.

In the drawings:

FIG. 1 shows the representative chemical finger prints of the structures of the major componenst of the dish-cultured fruit body (ARH003) and the cutting wood-cultured fruit body (ARH004) of Antrodia camphorata respectively; wherein the ethanol (95%) extract of the dish-cultured fruit body (ARH003) or the cutting wood-cultured fruit body (ARH004) were transferred to HPLC analysis and the profiles were recorded at UV 220 nm. The HPLC chromatograms of ARH003 (upper panel) and ARH004 (lower panel) were provided in FIG. 1.

FIG. 2 shows body weight (g) measurements of APP/PS1 mice with different genders and administration (of Vehicle or ARH003). APP/PS1 male (M) or female (F) mice (3 months old) orally administrated with vehicle (Veh) or 100 mg kg⁻¹day⁻¹ of ARH003 (A) for 4 months. The body weight changes were examined daily during ARH003 administration, and ARH003 significantly increased body weight in the final month of administration among male mice, but not among female mice. Significant differences between Veh group and ARH003 group are indicated by *, p<0.05.

FIG. 3A shows the representative fluorescent images of amylo-glo (white or blue), Iba-1 (red) and GFAP (green). APP/PS1 transgenic mice orally administered with vehicle and ARH003 for 4 months, and then amyloid plaques were stained by amylo-glo and microglia and astrocytes were immuno-stained with Iba-1 and GFAP antibodies, respectively. Scale bar: 1 mm.

FIG. 3B shows the magnification of typical plaque. Sale bar: 50 μm.

FIG. 3C shows the comparison between the number of amylo-glo-stained plaque in cerebral hemisphere without ARH003 administration (Veh) and with ARH003 administration (ARH003) after 4 months of administration. The results are the mean±S.E.M. Significant differences between Veh group and ARH003 group are indicated by *, p<0.05.

FIG. 3D shows the comparison between the number of activated astrocytes surround the plaque without ARH003 administration (Veh) and with ARH003 administration (ARH003) after 4 months of administration. Astrocytes were immuno-stained with GFAP antibody. The results are the mean±S.E.M.

FIG. 3E shows the comparison between the number of activated microglia surround the plaque without ARH003 administration (Veh) and with ARH003 administration (ARH003) after 4 months of administration. Microglia were immuno-stained with Iba-1 antibody. The results are the mean±S.E.M. Significant differences between Veh group and ARH003 group are indicated by ***, p<0.001.

FIG. 4A shows the results from burrowing task at 2 h and 16 h. APP/PS1 transgenic mice were orally administered with vehicle (Veh) or ARH003 (n=17 and 15 each). The tasks of burrowing were performed at 84^{th} days post administration. The results are the mean±S.E.M. Significant differences between Veh group and ARH003 group are indicated by **, p<0.01; ***, p<0.001.

FIG. 4B shows the comparison of the nesting task's nest score and unshredded Nestlet from nesting task. APP/PS1 transgenic mice were orally administered with vehicle (Veh) or ARH003 (n=17 and 15 each). The tasks of nesting were performed at 86^{th} days post administration. The results are the mean±S.E.M. Significant differences between Veh group and ARH003 group are indicated by **, p<0.01; ***, p<0.001.

FIG. 5A shows the representative swim paths in the hidden platform tests at 1s^{t} and last trial. APP/PS1 transgenic mice were orally administered with vehicle (Veh) or ARH003 (n=17 and 14 each). Morris water maze was performed.

FIG. 5B shows the comparison of the escape latency during the training phase. APP/PS1 transgenic mice were orally administered with vehicle (Veh) or ARH003 (n=17 and 14 each). Morris water maze (MWM) was performed. The results are the mean±S.E.M. Significant differences between Veh group and ARH003 group are indicated by *, p<0.01; **, p<0.01; ***, p<0.001.

FIG. 6A shows the representative swim path in a probe trial test. APP/PS1 transgenic mice were orally administered with vehicle (Veh) or ARH003 (n=17 and 14 each). Morris water maze was performed.

FIG. 6B shows the comparison of the latency to target-zone visit. APP/PS1 transgenic mice were orally administered with vehicle (Veh) or ARH003 (n=17 and 14 each). Morris water maze was performed. The results are the mean±S.E.M. Significant differences between Veh group and ARH003 group are indicated by ***, p<0.001.

FIG. 6C shows the comparison of the crossing times of the former platform. APP/PS1 transgenic mice were orally administered with vehicle (Veh) or ARH003 (n=17 and 14 each). Morris water maze was performed. The results are the mean±S.E.M.

### DETAILED DESCRIPTION OF THE INVENTION

Unless defined otherwise, all technical and scientific terms used herein have the same meaning as commonly understood by a person skilled in the art to which this invention belongs.

The present invention provides a method for treating dementia comprising administering a subject in need thereof an extract of Antrodia camphorata fruit body as an active ingredient.

Antrodia camphorata (AC), known in Taiwan as “niu Chang-chih” or “niu-chang-ku,” is a mushroom endemic to Taiwan (17). AC had long been popularly used as a folkloric medicine long before 1773, twisted tendon and muscle damages, terrified mental state, influenza and cold, headache, fever, and many internally affiliated diseases (18). Different extracts and compounds of AC have been found to exhibit various biological activities, including neuroprotective (19, 20), hepatoprotective, antihypertensive, anti-hyperlipidemic, anti-genotoxic, anti-angiogenic, antimicrobial, anti-cancer, anti-inflammatory, antioxidative, anti-viral, and immunomodulatory activities (21, 22).

Particularly, the extract of Antrodia camphorata fruit body is an extract of a dish cultured fruit body of Antrodia camphorate (ARH003) or an extract of a cutting wood-cultured fruit body of Antrodia camphorate (ARH004).

In the invention, the composition is proved to be effective for treating dementia, particularly AD.

Accordingly, the invention provides a use of the extract for manufacturing a medicament for treating dementia, particularly AD, particularly the extract of dish cultured Antrodia camphorata fruit body, i.e., ARH003 and/or ARH004.

In the invention, the extract ARH003/ARH004 comprises one or more compounds as active ingredients, which is selected from the group consisting of the following:

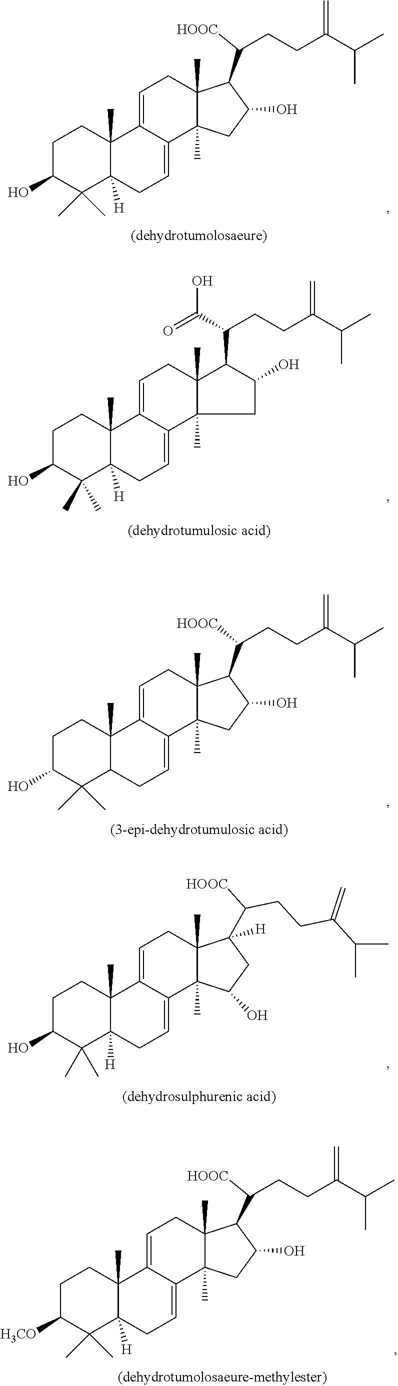

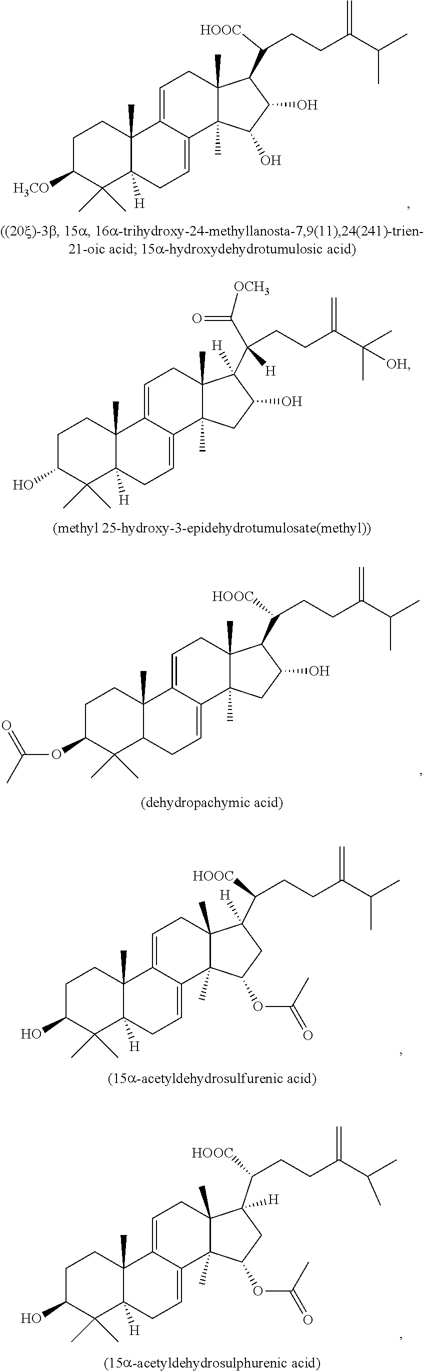

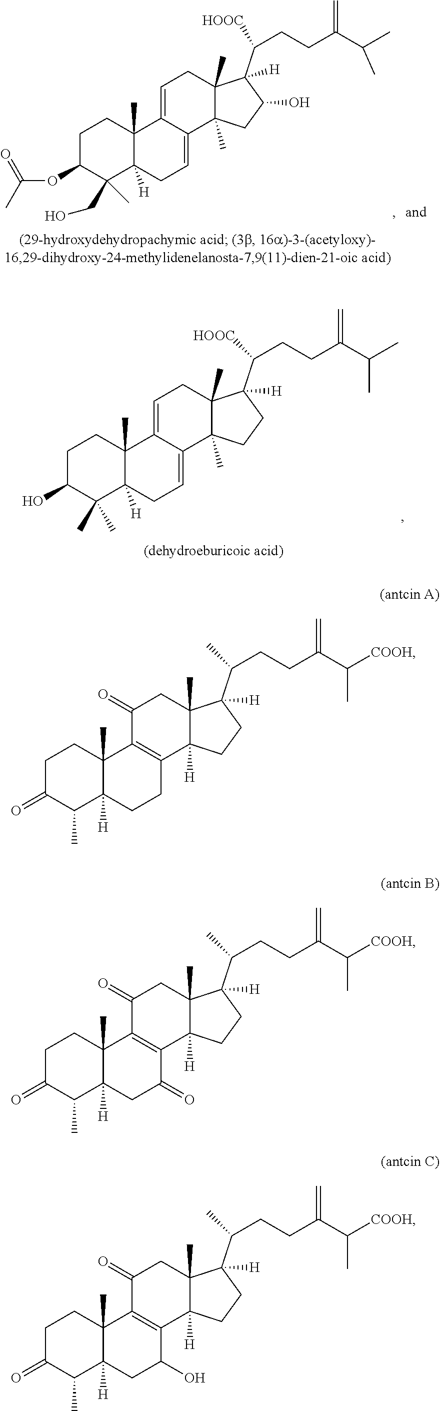

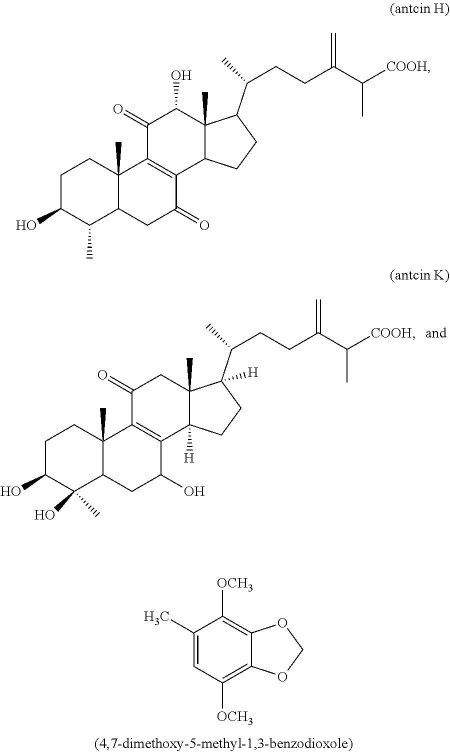 [SMILES: C=C(CC[C@@H](C)C1CCC2C3=C(C(=O)[C@H](O)[C@@]21C)[C@@]1(C)CC[C@H](O)[C@@H](C)C1CC3=O)C(C)C(=O)O ; C=C(CC[C@@H](C)[C@H]1CC[C@H]2C3=C(C(=O)C[C@]12C)[C@@]1(C)CC[C@H](O)[C@](C)(O)C1CC3O)C(C)C(=O)O ; COc1cc(C)c(OC)c2c1OCO2]

In preferred embodiments of the invention, the active compound is:

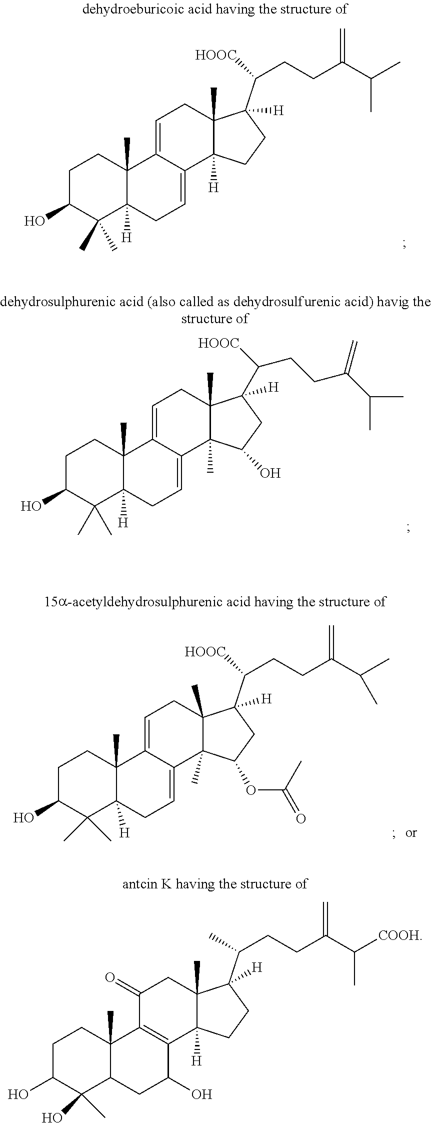

Accordingly, the present invention provides a method for treating dementia comprising administering to a subject in need there of an effective amount of the active compounds as mentioned above.

The term “therapeutically effective amount” as used herein refers to an amount of a pharmaceutical agent which, as compared to a corresponding subject who has not received such amount, results in an effect in treatment, healing, prevention, or amelioration of a disease, disorder, or side effect, or a decrease in the rate of advancement of a disease or disorder. The term also includes within its scope amounts effective to enhance normal physiological function.

For use in therapy, the therapeutically effective amount of the compound is formulated as a pharmaceutical composition for administration. Accordingly, the invention further provides a pharmaceutical composition comprising a therapeutically effective amount of ARH003 and/or ARH004 and one or more pharmaceutically acceptable carriers.

The term “pharmaceutically acceptable carrier” used herein refers to a carrier(s), diluent(s) or excipient(s) that is acceptable, in the sense of being compatible with the other ingredients of the formulation and not deleterious to the subject to be administered with the pharmaceutical composition. Any carrier, diluent or excipient commonly known or used in the field may be used in the invention, depending to the requirements of the pharmaceutical formulation. According to the invention, the pharmaceutical composition may be adapted for administration by any appropriate route, including but not limited to oral, rectal, nasal, topical, vaginal, or parenteral route. In one particular example of the invention, the pharmaceutical composition is formulated for oral administration. Such formulations may be prepared by any method known in the art of pharmacy.

The present invention is further illustrated by the following examples, which are provided for the purpose of demonstration rather than limitation.

### EXAMPLES

### Materials and Methods

1. Materials

BrdU, formic acid and Thioflavin S were purchased from Sigma-Aldrich (St Louis, Mo., USA). General chemicals were purchased from Sigma-Aldrich (St Louis, Mo., USA) or Merck (Darmstadt, Germany).

2. Extraction of Antrodia camphorata fruit body (Preparation of ARH003 and ARH004)

ARH003 was prepared from the dish cultured Antrodia camphorata. Fruiting body (300 g) and ARH004 was prepared from the cutting wood-cultured Antrodia camphorata (ARH004). The fruit body was heat refluxed with 95% ethanol. The ethanol solution was concentrated in vacuum to give a brown extract (60 g).

3. Management and Administration

The Institutional Animal Care and Use Committee at the National Research Institution of Chinese Medicine approved the animal protocol (IACUC No: 106-417-4). All experimental procedures involving animal and their care were carried out in accordance with Guide for the Care and Use of Laboratory Animals published by the United States National Institutes of Health (NIH). APP/PS1 was purchased from Jackson Laboratory (No. 005864). Breeding gender ratio was a male with two females in one cage. Experiments were conducted using wild type siblings and AD transgenic female C57BL/6J mice. The animals were housed under controlled room temperature (24±1° C.) and humidity (55-65%) with 12:12 h (07:00-19:00) light-dark cycle. All animal experimental procedures were performed based on Guide for the Care and Use of Laboratory Animals (NIH). APP/PS1 was purchased from Jackson Laboratory (No. 005864). Breeding were conducted using female transgenic mice and the male wild type siblings. The animals were housed under temperature (24±1° C.) and humidity (55-65%). Light-dark cycle was 12:12 h (07:00-19:00). All mice were provided with commercially available rodent normal chow diet and water ad libitum. For studying the therapeutic effect, both male and female APP/PS1 mice were administrated by oral gavage with vehicle (n=7 male and 8 female) or ARH003 or ARH004 (100 mg kg⁻¹ day⁻¹, n=7 male and 10 female) at 3 months of age for 4 months.

4. Tissue Processing

Mice after anesthetized were sacrificed by transcardial saline perfusion. Mice brain was removed and was immersed in 4% formaldehyde overnight at 4° C., and cryoprotected. Then brain tissue was sectioned into 30 μm thick. Three slides spanned approximately bregma −1.58 to −1.82 in each brain were used for staining and analysis.

5. Amylo-Glo Staining

Staining for fibrillar amyloid was performed either using Amylo-Glo as described by the manufacturer (Biosensis Inc., Thebarton, South Australia).

6. Immunohistochemistry

Immunohistochemistry was performed as described previously (39). Briefly, sections were blocked in phosphate buffered saline (PBS) containing 1% bovine serum albumin (BSA), 3% normal donkey serum and 0.3% Triton X-100 for 1 h. Then, incubated in PBS containing 1% BSA, 1% normal donkey serum, 0.3% Triton X-100, and primary antibodies, including mouse monoclonal antibodies to Aβ₁₋₁₆ (AB10, Millipore, MAB5208, 2757889), and glial fibrillary acidic protein (GFAP, Millipore, MAB5804, 1990686); and goat polyclonal antibody to anti-ionized calcium-binding adaptor molecule-1 (Iba-1) antibody (abcam, ab5076, GR268568-3) overnight at 4° C. Sections were then incubated in antibody dilution buffer containing Hoechst33258 (Invitrogen, 2 μg m⁻¹), Fluorescein isothiocyanate- or rhodamine red X (RRX)-conjugated donkey anti-mouse IgG, RRX-conjugated donkey anti-rabbit IgG or Alexa Fluor 647-conjugated donkey anti-goat IgG (Jackson ImmunoResearch, 705-605-147) at room temperature for 2 h. After washed with PBS containing 0.01% Triton X-100, sections were mounted with Aqua Poly/Mount (Polyscience Inc., Warrington, Pa., USA) for microscopic analysis using a Zeiss LSM 780 confocal microscopy (Jena, Germany). Representative confocal images were a 10-μm depth with maximal projection. Quantification of amyloid plaque was performed using ImageJ software. Amyloid plaque burden was calculated by the ratio of AB10-reactive or ThS-positive area to the total area.

7. Burrowing Test and Nesting Test

After oral gavage administration for 70 days, mice were assessed for burrowing test as described previously (46), with minor modifications. In brief, a practice run in group cage on day 70, and two individual tests on day 77 and 80 were performed. Mice were housed in new cage with a thin layer of bedding, and then a cylinder with 230 g of the food pellets was put into the cage at 16:00 next day. Finally, the remaining food pellets in cylinder were weighted after 2 h and overnight. The 2 h measurement of burrowed food pellets in the second individual test was exhibited in the results.

One day after burrowing test, nesting test was performed as described previously (35). In brief, two Nestlets (5 g) were placed into cage at 1 h before dark cycle, and then the nest score and the weight of unshredded Nestlets were determined after overnight. Nest construction was scored using a six-graded scale (40). A score of 0 indicates undisturbed Nestlet; 1, Nestlet was disturbed, but nesting material has not been gathered to a nest site in the cage; 2, a flat nest; 3, a cup nest; 4, an incomplete dome and 5, a complete and enclosed dome.

8. Morris Water Maze Test

After 90-day treatment, spatial memory performance was evaluated using a Morris water maze (MWM) test as described previously (41,42), with minor modifications. In briefly, the water maze apparatus consisted of a circular pool 120 cm in diameter, 40 cm deep, filled to the height of 20 cm with water (temperature 22-24° C.) to cover a platform (diameter 10 cm). The platform was submerged 1 cm below the surface of the opague water by adding non-toxic white paint. For descriptive data collection, four equal quadrants of the pool were conceptually divided. A computerized video imaging analysis system (Ethovision, Noldus Information Technology Inc, USA) was used to record the swimming paths of black mice in the white background of the maze.

Spatial memory test was conducted to study the spatial memory performance of the mice. All mice were trained in the MWM for 6 days. The platform was always placed in the center of the southwest quadrant. Each mouse was trained to find the platform with four trials a day with an inter-trial interval of 20 min. On each trial, the mouse was lowered gently into the water facing the pool wall at one of the three fixed locations according to a semi-random schedule. In case the mouse did not succeed within 60 s, it was aided onto the platform. At the conclusion of each trial, the mice were allowed to remain on the platform for 30 s whether it had found the platform or not. The escape latency to find the platform were measured in each trial and averaged over four trials.

A spatial probe test was performed wherein the extent of memory was assessed (43). The time spent in the target quadrant represented the degree of memory that had been obtained after learning during the training period. The 90-s probe trial (one trial without the platform) was assessed on the next day after 6-day acquisition training. The mouse was placed into the pool from the start location at the quadrant opposite of the former platform quadrant. The number of times the mouse crossed the former location of the platform and the time spent in the former platform quadrant were recorded for 90 s. The percentage of time spent in the center vs. periphery zones in the probe trial version of the MWM examined. The periphery zone is defined as the area between wall and the circle apart by 10-cm from the wall (44).

9. Statistical Analysis

The results are expressed as the mean±standard error of the mean (SEM) and processed for statistical analysis using GraphPad Prism 5 software. The parametric data were analyzed by unpaired two-tailed Student's t-test or one-way analysis of variance (ANOVA) with post-hoc Bonferroni's multiple comparisons tests. The nonparametric data, including crossing times of the platform in MWM test, amount of food pellets in burrowing test and nest score in nesting test, were analyzed using the Kruskal-Wallis ANOVA followed by post hoc Dunnett's multiple comparisons test.

### Example 1

### Molecular Characterization of ARH003 and ARH004 using HPLC Chromatography

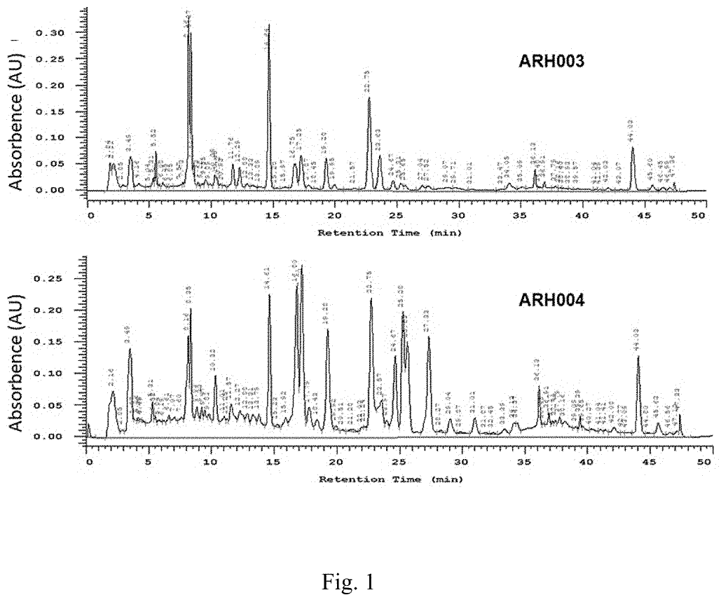

The Antrodia camphorata fruiting body used in this study is dish-cultured (ARH003) which posseses high phytomic similarity index with the Antrodia camphorata fruiting body cultured on Cinnamomum kenehirai wood (Chung et al., 2016). To confirm this similarity, HPLC chromatography was used to compare the components of dish-cultured Antrodia camphorata (ARH003) with the cutting wood-cultured Antrodia camphorata (ARH004) (FIG. 1).

### Example 2

### ARH003 Reduced Aβ Plaque Burden in Cerebrum of APP/PS1 Mice

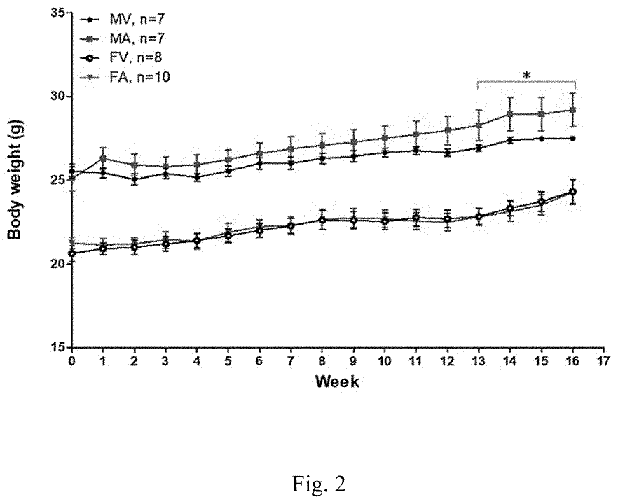

It is well established that plaques are visible in 6 months old APP/PS1 mice (23). Therefore, 3 months old APP/PS1 male or female mice orally administrated with vehicle or 100 mg kg⁻¹day⁻¹ of ARH003 for 4 months were employed to explore the effects of ARH003 on Aβ plaque deposition and glial cells activation. The body weight changes were examined during ARH003 administration, and ARH003 significantly increased body weight in the final month of administration among male mice, but not among female mice (FIG. 2).

### Example 3

### ARH003 Reduce the Number of Plaque with Glial Cluster in Cerebrum of APP/PS1 Mice

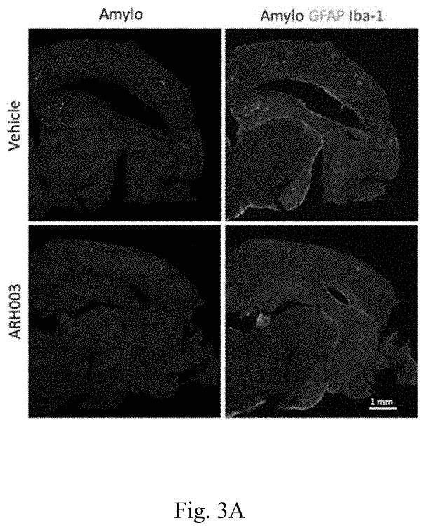

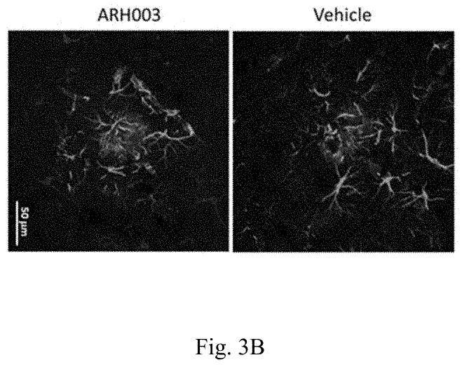

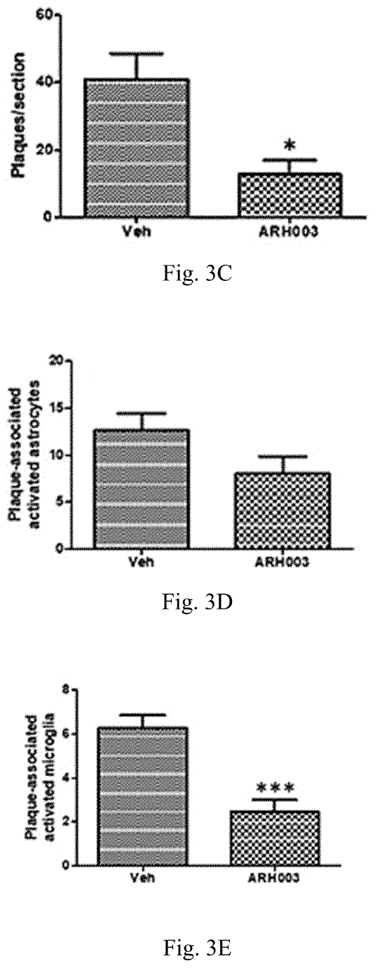

We evaluated the effects of ARH003 on the number of plaque with glial clusters. To determine plaque with glial clusters in cerebrum of APP/PS1 transgenic mice, Aβ plaque, microglia and astrocytes were examined by Amylo-glo staining and Iba-1- and GFAP-immunostaining, respectively. We found that the number of plaque with glial clusters decreased after the treatment of ARH003 (FIGS. 3A-3E).

### Example 4

### ARH003 Recovers the Cognitive Decline in APP/PS1 Mice

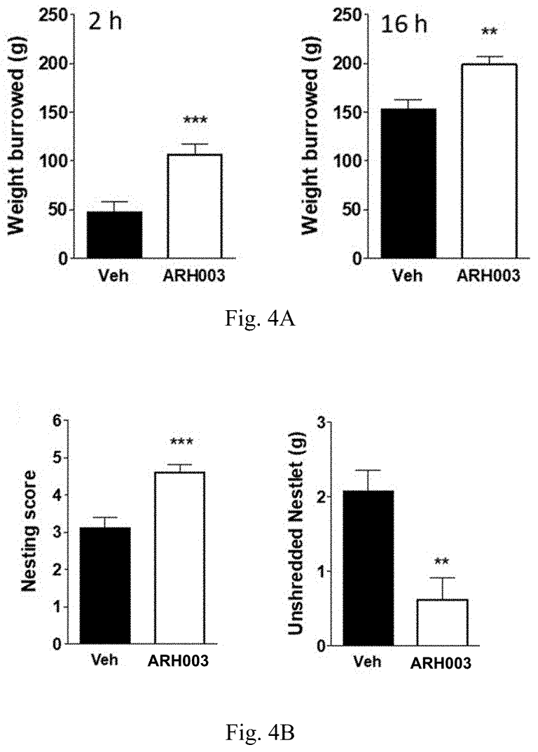

Burrowing and nesting behaviors, which engage a broad network of brain regions, have previously been applied on evaluating the ADL skills of AD transgenic mice. In the present study, three months old APP/PS1 mice were orally administered with ARH003 (100 mg kg⁻¹ day) for 114 days. The tasks of burrowing and nesting were then initiated at 84^{th} and 86th day after orally administration, respectively (FIG. 4A). APP/PS1 mice showed deficit in spontaneous burrowing behavior, and which was significantly recovered by administrating ARH003. APP/PS1 mice also showed deficit on nesting behavior evaluated by nest score and unshredded nestlet. The impaired nesting behavior was significantly recovered by administration of ARH003 (FIG. 4B).

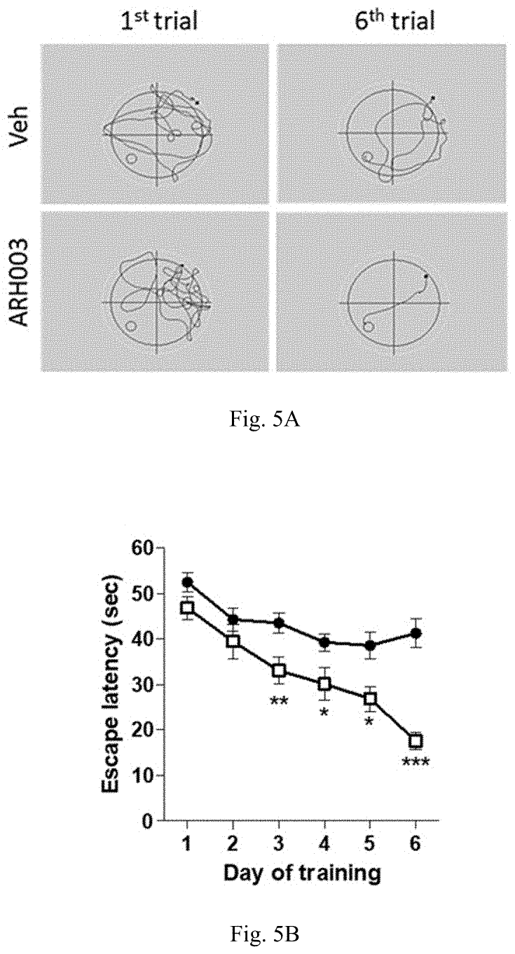

In MWM task, APP/PS1 mice showed a longer escape latency to find the hidden platform during the training phase, suggesting that APP/PS1 mice showed a spatial learning impairment at 7-month-old. This obvious impairment was significantly recovered by the treatment of ARH003 (FIGS. 5A-5B). Two-way repeated measurement ANOVA analysis confirmed an interaction between groups and days of training on escape latency to find the platform. It is significantly different among the days of training, among groups, and in subject. In Bonferroni posttests, It is significantly different between vehicle and ARH003 groups at 3th to 6th day of training.

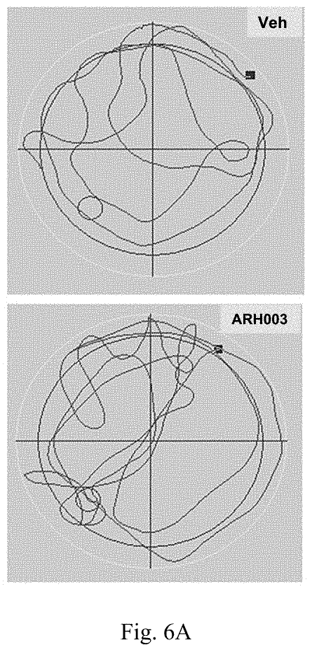

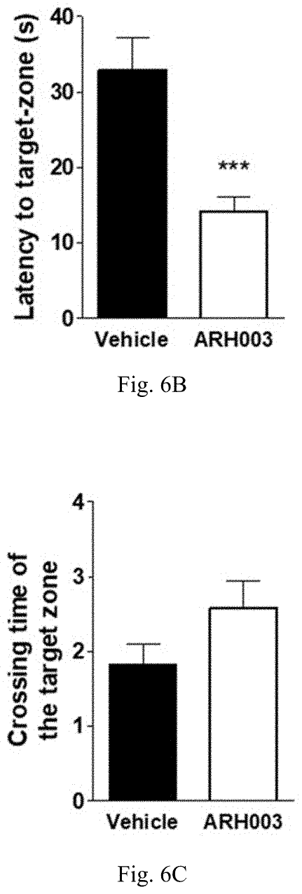

In probe trial, APP/PS1 mice showed reductions in latency to target zone visit and crossing time in target zone and (FIGS. 6A-6C) without affecting the swing speed (Data not shown). Again, this impairment was significantly recovered by the treatment of ARH003.

### REFERENCES

1. Hardy, J.; Selkoe, D. J. The amyloid hypothesis of Alzheimer's disease: progress and problems on the road to therapeutics. Science. 2002, 297, 353-356.

2. Fuster-Matanzo, A.; Llorens-Martin, M.; Hernández, F.; Avila, J. Role of neuroinflammation in adult neurogenesis and Alzheimer disease: therapeutic approaches. Mediators Inflamm. 2013, 2013, 260925.

3. Winner, B.; Winkler, J. Adult neurogenesis in neurodegenerative diseases. Cold Spring Harb. Perspect Biol. 2015, 7, a021287.

4. Selkoe, D. J. Clearing the brain's amyloid cobwebs. Neuron. 2011, 232, 177-180.

5. Ryan, S. M.; Nolan, Y. M. Neuroinflammation negatively affects adult hippocampal neurogenesis and cognition: can exercise compensate? Neurosci. Biobehav. Rev. 2015, 61, 121-131.

6. Lee, I. S.; Jung, K.; Kim, I. S.; Park, K. I. Amyloid-β oligomers regulate the properties of human neural stem cells through GSK-3β signaling. Exp. Mol. Med. 2013, 45, e60.

7. Nicolas, M.; Hassan, B. A. Amyloid precursor protein and neural development. Development. 2014, 141, 2543-2548.

8. Sierra, A.; Beccari, S.; Diaz-Aparicio, I.; Encinas, J. M.; Comeau, S.; Tremblay, M, Ė. Surveillance, phagocytosis, and inflammation: how never-resting microglia influence adult hippocampal neurogenesis. Neural Plast. 2014, 2014, 610343.

9. Capsoni, S.; Tiveron, C.; Vignone, D.; Amato, G.; Cattaneo, A. Dissecting the involvement of tropomyosin-related kinase A and p75 neurotrophin receptor signaling in NGF deficit-induced neurodegeneration. Proc. Natl. Acad. Sci. USA. 2010, 107, 12299-12304.

10. Zhang, H.; Petit, G. H.; Gaughwin, P. M.; Hansen, C.; Ranganathan, S.; Zuo, X.; Smith, R.; Roybon, L.; Brundin, P.; Mobley, W. C.; Li. J. Y. NGF rescues hippocampal cholinergic neuronal markers, restores neurogenesis, and improves the spatial working memory in a mouse model of Huntington's Disease. J. Huntingtons Dis. 2013, 2, 69-82.

11. Jankowsky, J. L.; Fadale, D. J.; Anderson, J.; Xu, G. M.; Gonzales. V; Jenkins, N. A.; Copeland, N. G.; Lee, M. K.; Younkin, L. H.; Wagner, S. L.; Younkin, S. G.; Borchelt, D. R. Mutant presenilins specifically elevate the levels of the 42 residue beta-amyloid peptide in vivo: evidence for augmentation of a 42-specific gamma secretase. Hum. Mol. Genet. 2004, 13, 159-170.

12. Malm, T.; Koistinaho, J.; Kanninen, K. Utilization of APPswe/PS1dE9 Transgenic Mice in Research of Alzheimer's Disease: Focus on Gene Therapy and Cell-Based Therapy Applications. Int. J. Alzheimers Dis. 2011, 2011, 517160.

13. Volianskis, A.; Kostner. R.; Molgaard, M.; Hass, S.; Jensen, M. S. Episodic memory deficits are not related to altered glutamatergic synaptic transmission and plasticity in the CA1 hippocampus of the APPswe/PS1deltaE9-deleted transgenic mice model of β-amyloidosis. Neurobiol. Aging 2010, 31, 1173-1187.

14. Reiserer, R. S.; Harrison, F. E.; Syverud, D. C.; McDonald, M. P. Impaired spatial learning in the APPSwe+PSEN1DeltaE9 bigenic mouse model of Alzheimer's disease. Genes Brain Behav. 2007, 6, 54-65.

15. Xiong, H.; Callaghan, D.; Wodzinska, J.; Xu, J.; Premyslova, M.; Liu, Q. Y.; Connelly, J.; Zhang, W. Biochemical and behavioral characterization of the double transgenic mouse model (APPswe/PS1dE9) of Alzheimer's disease. Neurosci. Bull. 2011, 27, 221-232.

16. Hamilton, A.; Holscher, C. The effect of ageing on neurogenesis and oxidative stress in the APP(swe)/PS1(deltaE9) mouse model of Alzheimer's disease. Brain Res. 2012, 1449, 83-93.

17. Wu, S H.; Ryvarden L,; Chang, T. T. Antrodia camphorata (“niu-chang-chih”), new combination of a medicinal fungus in Taiwan. Bot. Bull. Acad. Sin. 1997, 38, 273-275.

18. Peng, C. C.; Chen, K. C.; Peng, R. Y.; Chyau, C. C.; Su, C. H.; Hsieh-Li, H. M. Antrodia camphorata extract induces replicative senescence in superficial TCC, and inhibits the absolute migration capability in invasive bladder carcinoma cells. J. Ethnopharmacol. 2007, 109, 93-103.

19. Chen, C. C.; Shiao, Y. J.; Lin, R. D.; Shao, Y. Y.; Lai, M. N.; Lin, C. C.; et al., Neuroprotective diterpenes from the fruiting body of Antrodia camphorata. J. Nat. Prod. 2006, 69, 689-691.

20. Phan, C. W.; David, P.; Naidu, M.; Wong, K. H.; Sabaratnam, V. Therapeutic potential of culinary-medicinal mushrooms for the management of neurodegenerative diseases: diversity, metabolite, and mechanism. Crit. Rev. Biotechnol. 2015, 35, 355-368.

21. Yue, P. Y.; Wong, Y. Y.; Wong, K. Y.; Tsoi, Y. K.; Leung, K. S. Current evidence for the hepatoprotective activities of the medicinal mushroom Antrodia cinnamomea. Chin. Med. 2013,8, 21.

22. Geethangili, M.; Tzeng, Y. M. Review of Pharmacological Effects of Antrodia camphorata and Its Bioactive Compounds. Evid. Based Complement. Alternat. Med. 2011, 2011,212641.

23. Yan, P.; Hu, X.; Song, H.; Yin, K.; Bateman, R. J.; Cirrito, J. R.; Hsu, F. F.; Turk, J. W.; Xu, J.; Hsu, C. Y.; Holtzman, D. M.; Lee, J. M. Matrix metalloproteinase-9 degrades amyloid-beta fibrils in vitro and compact plaques in situ. J. Biol. Chem. 2006, 281, 24566-24574.

24. Vepsäläinen, S.; Koivisto, H.; Pekkarinen, E.; Mäkinen, P.; Dobson, G.; McDougall, G. J.; Stewart, D.; Haapasalo, A.; Karjalainen, R. O.; Tanila, H.; Hiltunen, M. Anthocyanin-enriched bilberry and blackcurrant extracts modulate amyloid precursor protein processing and alleviate behavioral abnormalities in the APP/PS1 mouse model of Alzheimer's disease. J. Nutr. Biochem. 2013, 24, 360-370.

25. Mawuenyega, K. G.; Sigurdson, W.; Ovod, V.; Munsell, L.; Kasten, T.; Morris, J. C.; Yarasheski, K. E.; Bateman, R. J. Decreased clearance of CNS beta-amyloid in Alzheimer's disease. Science. 2010, 330, 1774.

26. Qiu, W. Q.; Folstein, M. F. Insulin, insulin-degrading enzyme and amyloid-beta peptide in Alzheimer's disease: review and hypothesis. Neurobiol. Aging. 2006, 27, 190-198.

27. Baranello, R. J.; Bharani, K. L.; Padmaraju, V.; Chopra, N.; Lahiri, D. K.; Greig, N. H.; Pappolla, M. A.; Sambamurti, K. Amyloid-beta protein clearance and degradation (ABCD) pathways and their role in Alzheimer's disease. Curr. Alzheimer Res. 2015, 12, 32-46.

28. Serrano-Pozo, A.; Muzikansky, A.; Gómez-Isla, T.; Growdon, J. H.; Betensky, R. A.; Frosch, M. P.; Hyman, B. T. Differential relationships of reactive astrocytes and microglia to fibrillar amyloid deposits in Alzheimer disease. J. Neuropathol. Exp. Neurol. 2013, 72, 462-471.

29. Fu, A. K.; Hung, K. W.; Yuen, M. Y.; Zhou, X.; Mak, D. S.; Chan, I. C.; Cheung, T. H.; Zhang, B.; Fu, W. Y.; Liew, F. Y.; Ip, N. Y. IL-33 ameliorates Alzheimer's disease-like pathology and cognitive decline. Proc. Natl. Acad. Sci. USA. 2016, 113, E2705-E2713.

30. El Khoury, J.; Toft, M.; Hickman, S. E.; Means, T. K.; Terada, K.; Geula, C.; Luster, A. D. Ccr2 deficiency impairs microglial accumulation and accelerates progression of Alzheimer-like disease. Nat. Med. 2007, 13, 432-438.

31. Goto, Y.; Niidome, T.; Hongo, H.; Akaike, A.; Kihara, T.; Sugimoto, H. Impaired muscarinic regulation of excitatory synaptic transmission in the APPswe/PS1dE9 mouse model of Alzheimer's disease. Eur. J. Pharmacol. 2008, 583, 84-91.

32. Yeh, C. W.; Yeh, S. H.; Shie, F. S.; Lai, W. S.; Liu, H. K.; Tzeng, T. T.; Tsay, H. J.; Shiao, Y. J. Impaired cognition and cerebral glucose regulation are associated with astrocyte activation in the parenchyma of metabolically stressed APPswe/PS1dE9 mice. Neurobiol. Aging 2015, 36, 2984-2994.

33. Duan, S.; Guan, X.; Lin, R.; Liu, X.; Yan, Y.; Lin, R.; Zhang, T.; Chen, X.; Huang, J.; Sun, X.; Li, Q.; Fang, S.; Xu, J.; Yao, Z.; Gu, H. Silibinin inhibits acetylcholinesterase activity and amyloid β peptide aggregation: a dual-target drug for the treatment of Alzheimer's disease. Neurobiol. Aging 2015, 36, 1792-1807.

34. Deacon, R. M. Burrowing in rodents: a sensitive method for detecting behavioral dysfunction. Nat. Protoc. 2006, 1, 118-121.

35. Deacon, R. M. Assessing nest building in mice. Nat. Protoc. 2006, 1, 1117-1119.

36. Jirkof, P. Burrowing and nest building behavior as indicators of well-being in mice. J. Neurosci. Meth. 2014, 234, 139-146.

37. Woodbridge, R.; Sullivan, M. P.; Harding, E.; Crutch, S.; Gilhooly, K. J.; Gilhooly, M.; McIntyre, A.; Wilson, L. Use of the physical environment to support everyday activities for people with dementia: A systematic review. Dementia (London). 2016, pii: 1471301216648670.

38. Janus, C.; Flores, A. Y.; Xu, G.; Borchelt, D. R. Behavioral abnormalities in APPSwe/PS1dE9 mouse model of AD-like pathology: comparative analysis across multiple behavioral domains. Neurobiol. Aging. 2015, 36, 2519-2532.

39. Tsai-Teng, T.; Chin-Chu, C.; Li-Ya, L.; Wan-Ping, C.; Chung-Kuang, L.; Chien-Chang, S.; Chi-Ying, H. F.; Chien-Chih, C.; Shiao, Y. J. Erinacine A-enriched Hericium erinaceus mycelium ameliorates Alzheimer's disease-related pathologies in APPswe/PS1dE9 transgenic mice. J. Biomed. Sci. 2016, 23, 49.

40. Hess, S. E.; Rohr, S.; Dufour, B. D.; Gaskill, B. N.; Pajor, E. A.; Garner, J. P. Home improvement: C57BL/6J mice given more naturalistic nesting materials build better nests. J. Am. Assoc. Lab. Anim. Sci. 2008, 47, 25-31.

41. Morris, R. Developments of a water-maze procedure for studying spatial learning in the rat. J. Neurosci. Methods. 1984, 11, 47-60.

42. Chao, H. W.; Tsai, L. Y.; Lu, Y. L.; Lin, P. Y.; Huang, W. H.; Chou, H. J.; Lu, W. H.; Lin, H. C.; Lee, P. T.; Huang, Y. S. Deletion of CPEB3 enhances hippocampus-dependent memory via increasing expressions of PSD95 and NMDA receptors. J. Neurosci. 2013, 33, 17008-17022.

43. Wang, L. S.; Zhou, J.; Shao, X. M.; Tang, X. C. Huperzine A attenuates cognitive deficits and brain injury in neonatal rats after hypoxia-ischemia. Brain Res. 2002, 949, 162-170.

44. Kim, T. K.; Han, H. E.; Kim, H.; Lee, J. E.; Choi, D.; Park, W. J.; Han, P. L. Expression of the plant viral protease NIa in the brain of a mouse model of Alzheimer's disease mitigates Abeta pathology and improves cognitive function. Exp. Mol. Med. 2012, 44, 740-748.

What is claimed is:

1. A method for treating dementia comprising administering a subject in need thereof an effective amount of an extract of Antrodia camphorata fruit body.

2. The method of claim 1, wherein the extract of Antrodia camphorata fruit body is the extract of a dish cultured fruit body thereof.

3. The method of claim 1, wherein the extract of Antrodia camphorata fruit body is the extract of a cutting wood-cultured fruit body thereof.

4. The method of claim 1, wherein the extract of Antrodia camphorate fruit body is prepared through extraction of Antrodia camphorate fruit body with water or an organic solvent.

5. The method of claim 4, wherein the organic solvent is ethanol.

6. A method for treating dementia, comprising administering a subject in need thereof an effective amount of a compound selected from the group consisting of: 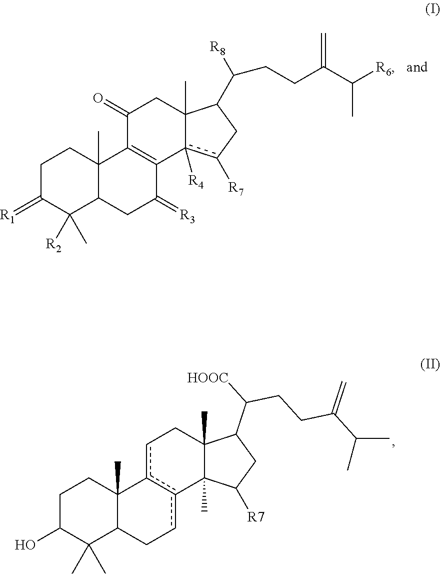
wherein R₁ is O, α-OH or β-H; R₂ is H or OH; R₃ is O, α-H, β-OAc or H₂; R₄ is H or OH; R₅ is H or OH; R₆ is COOH or COO(CH₂)n-CH₃; R₇ is H, OH, or OAc; R₈ is CH₃ or COOH; the dotted line represents a single bond or a double bond; n is an integer from 0-3.

7. A method for treating dementia, comprising administering a subject in need thereof an effective amount of a compound selected from the group consisting of:
dehydroeburicoic acid having the structure of:

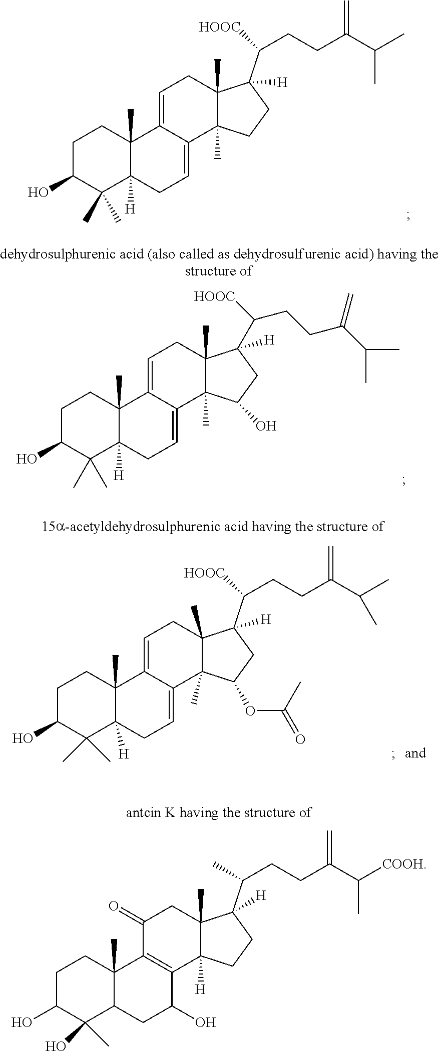

8. The method of claims 6, wherein the dementia is Alzheimer's disease (AD).

9. The method of claims 7, wherein the dementia is Alzheimer's disease (AD).

10. The method of claims 1, in which the extract is effective in suppressing Aβ plaque deposition and glial cells activation.

11. The method of claim 1, in which the extract is effective in improving memory impairments.

12. The method of claim 6, in which the compound is effective in suppressing Aβ plaque deposition and glial cells activation.

13. The method of claim 7, in which the compound is effective in suppressing Aβ plaque deposition and glial cells activation.

14. The method of claims 6, in which the compound is effective in improving memory impairments.

15. The method of claims 7, in which the compound is effective in improving memory impairments.
